(19) United States
(12) Patent Application Publication
(10) Pub. No.: US 2001/0007743 A1
(43) Pub. Date: Jul. 12, 2001

(54) METHOD FOR SCREENING COMPOUNDS WHICH INTERACT WITH THE L-ENANTIOMER OF A TARGET RNA

(76) Inventors: DAVID JUSTIN CHARLES KNOWLES, NORTH YORKSHIRE (GB); BRIAN METCALF, RADNOR, PA (US); NEVILLE HUBERT NICHOLSON, SUFFOLK (GB); NEIL DAVID PEARSON, HERTFORDSHIRE (GB); CATHERINE DENISE PRESCOTT, CAMBRIDGESHIRE (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 09/180,370

(22) PCT Filed: May 8, 1997
(86) PCT No.: PCT/GB97/01236

§ 371 Date: None

(30) Foreign Application Priority Data
May 9, 1996 (GB) 9609722.5

(51) Int. Cl. G01N 33/53; C12Q 1/68; C12P 19/34; C07H 21/04

(52) U.S. Cl. 435/6; 435/7.1; 435/91.2; 536/23.1

(57) ABSTRACT

A method of identifying compounds which interact, directly or indirectly, with a target molecule of interest, wherein the target molecule of interest is an RNA, comprises screening compounds for interaction with the (L)-enantiomer of the said RNA, and identifying, as hits, the enantiomers of those compounds for which a said interaction is detected.

This invention relates to a method of screening compounds in order to identify those having a desired biological activity.

It is usual for new leads for drug discovery to be generated by screening of single synthetic compounds made individually in the laboratory or by screening extracts obtained from natural product sources such as microbial metabolites, marine sponges and plants.

A more recent approach to lead generation has been high throughput screening of groups of compounds against biological targets. These groups may be assembled from collections of compounds previously individually prepared and since stored in a compound bank, the assembly being random or guided by the use of ‘diversity’ programmes. In addition, there has also been a rapid growth in the deliberate preparation and use of so-called libraries. Each library contains a large number of compounds which are screened against a whole array of biological targets. When a ‘hit’ is found, the library is ‘deconvoluted’, so that the member(s) thereof responsible for the ‘hit’ can be identified. It is expected that the lead would have relatively weak activity in the screen but would then form the basis of a more traditional medicinal chemistry programme, to enhance activity. The libraries may be prepared by the use of the so-called ‘combinatorial’ and/or ‘variomer’ approaches (Jung et al, Angew Chem Int Ed Engl, 31:367-83, 1992; Pavia et al, Bioorg Med Chem Lett, 3:387-96, 1993).

In the combinatorial approach, several variations of the same reaction are carried out on a substrate, in parallel and in the same reaction vessel, to provide a mixture of compounds or a combinatorial library. The first combinatorial libraries were composed of small polypeptides, with some libraries containing up to 10,000 members. Such libraries could be made using the techniques developed for the synthesis of single polypeptides (see, for instance, Lam et al, Nature, 354: 82, 1991 and WO 92/00091; Geysen et al, J Immunol Meth, 102: 259, 1987; and Houghten et al, Nature, 354: 84, 1991 and WO 92/09300). A first amino acid, normally attached to a resin support, is allowed to react with a number of different amino acids, for instance 5, to produce 5 different dipeptides. This mixture of dipeptides can then be reacted with another 5 different amino acids, to produce 25 different tripeptides. This process can be repeated several times over, to give libraries of ever increasing number of different polypeptides.

Another method of preparing a library of peptides for screening purposes is by means of phage display (sometimes referred to as “combinatorial biology”). Phage display libraries are described by Scott et al (Science 249 386 (1990)), Devlin et al (ibid p404), and Cwirla et al (Proc. Natl. Acad. Sci. USA 87 6378 (1990)).

Methods have also been described for preparing libraries of small non-peptide molecules based upon a common template or core structure (see for instance Ellman and Bunin, J Amer Chem Soc, 114:10997, 1992 (benzodiazepine template) and WO 94/00509 (oxazolone template)). The template will have a number of functional sites, for instance three, each of which can be reacted, in a stepwise fashion, with a number of different reagents, for instance five, to introduce 5×5×5 different combinations of substituents, giving a library containing 125 components. The library will normally contain all or substantially all possible permutations of the substituents.

The biological assays which are used to identify compounds having the desired biological activity rely on detecting a direct or indirect interaction between the compound to be screened and a target molecule which is usually of natural origin and comprises for example a protein (such as a receptor or a bacterial or viral protein) or a nucleic acid or a fragment thereof.

An example of a biological assay is described by Laird-Offringa et al (Proc. Natl. Acad. Sci USA 92 11859-11863 (1995)). In this paper, the RNA-binding domain of the mammalian splicesomal protein U1A was randomly mutagenised and displayed as a combinatorial library on filamentous bacteriophage. An RNA fragment which the protein binds to was synthesised and immobilised by annealing it via a 3′-tail complementary to a 5′-biotinylated oligodeoxynucleotide. Affinity selection was used to identify residues responsible for strong binding.

In that example, the target molecule was an RNA fragment, and the interaction which was being detected was a direct one between the target molecule and the peptides being screened. Mei et al (Chem. Letts 5 2755 (1995)) describe a method in which the binding of 12 mer and 40 mer peptides (fragments of the HIV tat protein) to a 31 nucleotide fragment of TAR RNA was studied in the presence and absence of aminoglycosides. They were found to inhibit protein binding to the RNA. Dissociation constants were obtained for the peptides and IC₅₀ values for the added aminoglycosides. The tat/TAR interaction is important for HIV replication. This is an example of an indirect interaction between compounds to be screened and the target molecule.

WO 94/09792 describes the screening of a total of thirty two antibiotics for their ability to inhibit tat binding (see Example 10). A 26-nucleotide ³²P-labelled TAR RNA probe (bp18 to 44) was incubated with a synthetic peptide containing amino acids 48-60 of the HIV-1 tat protein, in the presence or absence of 250 micromoles of the antibiotic to be tested. Binding was quantified by an RNA gel mobility shift assay.

One disadvantage of currently available screening methods is that the RNAs used as target molecules are susceptible to enzymatic degradation and therefore cannot be used in screening of natural product extracts, which are enzymatically active. Furthermore, peptides which have been identified using the above-mentioned methods as having useful biological activity may nevertheless not be pharmaceutically useful because they are degraded by enzymes in vivo or because they induce an immune response that impairs their activity.

Schumacher et al (Science 271 1854-1857 (1996)) describe a method in which a phage display library was screened against a protein subdomain of interest which had been synthesised in the (D)-amino acid configuration, ie the non-naturally occurring configuration. The (D)-enantiomer of one of the peptides which were identified as binding to this subdomain was then synthesised and found to bind as predicted to the naturally occurring form of the subdomain of interest.

According to the present invention, there is provided a method of identifying compounds which interact, directly or indirectly, with a target molecule of interest, wherein the target molecule of interest is an RNA, characterised in that the method comprises screening compounds for interaction with the (L)-enantiomer of the said RNA, and identifying, as hits, the enantiomers of those compounds for which a said interaction is detected.

One advantage of the method of the present invention is that, in combination with screening with natural RNA, it effectively doubles the number of compounds that can be screened at any one time, and also makes available for screening compounds which would not normally be available, such as the enantiomers of chiral products from nature and of peptides expressed by a phage display library, or which would be difficult to make, such as the enantiomers of a combinatorial library for which only a single handed template is available.

Another advantage is that the (L)-RNA used in carrying out the invention is not susceptible to enzymatic degradation and can therefore be used in the screening of natural product extracts. Similarly, peptides identified using this method are in the (D)-configuration and therefore may have an improved pharmacokinetic profile.

The target molecule comprises for example the whole or a fragment of a naturally-occurring RNA, such as an RNA of human, bacterial, or viral origin.

The compounds to be screened may be individual synthetic compounds or may be present in a natural product extract or form part of a compound bank, a phage display library or a combinatorial library.

The interaction to be detected may be a direct interaction such as binding of the compound to the (L)-RNA or an indirect interaction such as inhibition of binding of the (L)-RNA to another molecule.

The other molecule may for example be the enantiomer of a protein or protein fragment to which the target molecule RNA normally binds.

A further aspect of the invention provides compounds identified using the method of the invention, as well as their use in therapy.

A still further aspect of the invention provides a novel compound which inhibits the function of a target RNA, characterised in that the compound is the enantiomer of a compound found in nature.

The compound may for example inactivate gene expression by binding the target RNA or by inhibiting its binding to a regulatory protein.

Enantiomers of compounds extracted from nature have not previously been synthesised for screening purposes because of the large number of compounds which need to be screened in order to obtain a “hit”, making such an exercise not cost effective. The method of the present invention enables large numbers of compounds to be screened before they are synthesised.

### EXAMPLES

### Example 1

Preparation of (L)-RNA

(a) 5′-O-(4,4′-Dimethoxytrityl)-2′-O-(tert-butyldimethylsilyl)-(L)-uridine-3′-(O-cyanoethyl-N,N-diisopropylphosphoramidite.

The title compound is prepared as described by Ashley, G. W. (J. Amer. Chem. Soc. 114 9731 (1995)) from (L)-uridine (Holy et al, Collect. Czech. Chem. Commun. 36 3282 (1971)).

The cytosine, adenosine, and guanosine analogues are prepared from (L)-cytosine, (L)-adenosine and (L)-guanosine (Holy et al, ibid; Visser et. al Recl. Trav. Chim. Pays-Bas 105 528 (1986)) using the methods described by Beaucage et al (Tetrahedron 48 2223-2311 (1992)) for the (D)-analogues.

(b) Synthesis of (L)-RNA.

The RNA is synthesised as described by Gait et al (Oligonucleotides and analogues, Ed. F. Eckstein, IRL Press, 1991, p25) using phosphoramidite chemistry. This is automated or carried out using the syringe technique (Ashley et al, J. Amer. Chem. Soc. 114 9731 (1995)). The oligoribonucleotides may also be made by H phosphonate chemistry, by coupling of phosphate acids with the alcohol, or by another of the chemistries described by Beaucage et al. Modified nucleotides can also be incorporated. (L)-RNA synthesis is also described by Klussmann et al, Nolte et al (infra).

Where the size of the RNA is greater than 20 nts, the use of photocleavable biotin derivatives as described by Olejnik et. al. (Nucleic Acids Research 24 361 (1996)) can be used to aid the synthesis and purification of oligonucleotides, providing 5′-phosphorylated oligonucleotides. These can then be used in screening methods instead of the 5′-non-phosphorylated analogues.

### Example 2

Preparation of (D)-peptides

Protected (D)-amino acids are commercially available from Bachem California, Bachem Biosciece, Advanced Chemtech, and Novabiochem. For (D)-Ile and (D)-Thr, the chirality of the side chain is also inverted relative to (L)-Ile and (L)Thr. Synthesis of (D)-peptides is carried out conventionally using a peptide synthesiser. See for example “Solid phase peptide synthesis” by J. M. Stewart and J. D. Young, 1984, second edition, Pierce Chemical Company, Rockford, Ill., and “Solid phase peptide synthesis” by E. Atherton and R. C. Sheppard, 1989, IRL Press, Oxford.

### Example 3

Screening methods that use RNA as a target molecule

As described in WO 94/09792 compounds were screened for their ability to inhibit tat binding (see Example 10). A 26-nucleotide ³²P-labelled TAR RNA probe (bp18 to 44) was incubated with a synthetic peptide containing amnino acids 48-60 of the HIV-1 tat protein, in the presence or absence of 250 micromoles of the compound to be tested. Binding was quantified by an RNA gel mobility shift assay.

As described in WO 94/29487 and WO 94/09792 compounds were screened for their ability to inhibit Rev binding. For example, a 47-nucleotide ³²P-labelled RRE RNA probe (a IIB RNA) was incubated with a synthetic peptide suc-Rev₃₄₋₅₀ AAAAR-am peptide derived from the HIV-1 Rev protein, in the presence or absence of 250 micromoles of the compound to be tested. Binding was quantified by an RNA gel mobility shift assay.

Alternatively, other peptides derived from Rev, as described in WO 94/29487, and other RNAs derived from RRE (e.g. RREIIB-TR, 34 nts, as described by Battiste et. al, Biochemistry 33 2741(1994)) can be used.

### Example 4

Resistance of (L)-RNA to naturally-occurring ribonucleases

A 58-mer (D)-RNA and its enantiomer (L)-RNA, designated D-A42d and L-A42d respectively, were prepared as described in Nature Biotechnology 14 September 1996, 1112-1119 (Klussmann et al, Nolte et al), and subjected to digestion in human serum. Whilst D-A42d was rapidly degraded, L-A42d showed no evidence of degradation after a 60-h incubation (FIG. 5, ibid).

1. A method of identifying compounds which interact, directly or indirectly, with a target molecule of interest, wherein the target molecule of interest is an RNA, characterised in that the method comprises screening compounds for interaction with the (L)-enantiomer of the said RNA, and identifying, as hits, the enantiomers of those compounds for which a said interaction is detected.

2. A method according to claim 1 wherein the target molecule comprises the whole or a fragment of a naturally-occurring RNA of human, bacterial, or viral origin.

3. A method according to claim 1 or 2, wherein the compounds to be screened are individual synthetic compounds or compounds present in a natural product extract, or form part of a compound bank, a phage display library or a combinatorial library.

4. A method according to any one of the preceding claims, wherein the interaction to be detected is binding of the compound to the (L)-RNA or inhibition of binding of the (L)-RNA to another molecule.

5. A method according to claim 4, wherein the other molecule is the enantiomer of a protein or protein fragment to which the target molecule RNA normally binds.

6. A compound identified using the method of any one of the preceding claims.

7. A compound according to claim 6, for use in therapy.

8. A novel compound which inhibits the function of a target RNA, characterised in that the compound is the enantiomer of a compound found in nature.
